(19) United States
(12) Patent Application Publication
(10) Pub. No.: US 2016/0045098 A1
(43) Pub. Date: Feb. 18, 2016

(54) HEART INNER WALL CHECKING TOOL AND DEVICE FOR CHECKING HEART INNER WALL

(71) Applicant: Thoratec Corporation, Pleasanton, CA (US)

(72) Inventor: Takeshi Tsubouchi, Kanagawa (JP)

(21) Appl. No.: 14/761,146

(22) PCT Filed: Jan. 18, 2013
(86) PCT No.: PCT/JP2013/050941

§ 371 Date: Nov. 4, 2015

(51) Int. Cl. A61B 1/00; A61B 1/313; A61B 1/015; A61B 1/05

(52) U.S. Cl. CPC A61B 1/00082; A61B 1/00154; A61B 1/05; A61B 1/00165; A61B 1/3137; A61B 1/015; A61B 1/00124; A61B 1/00096; A61M 2025/1061

(57) ABSTRACT

A heart inner wall checking device 100 comprises heart inner wall checking tool 1, and shaft-shaped imaging tool 5, which can be inserted into heart inner wall checking tool 1. Heart inner wall checking tool 1 comprises tubular main body 2, and an inflatable balloon 3, which is provided on the distal end of tubular main body 2. Heart inner wall checking tool 1 also comprises liquid injection port 42 for injecting an inflation liquid into balloon 3 and imaging member port 43 for inserting shaft-shaped imaging member 5, with which a rear side area can be checked. Balloon 3 has transparency, which enables imaging of the outside of balloon 3 with area image sensor 51, and includes balloon rear portion 32, which is capable of coming into close contact with the heart inner wall during inflation. Balloon rear portion 32 includes liquid-exudable member 33, which exudes the inflation liquid and supplies the inflation liquid between balloon rear portion 32 and heart inner wall 12.

### TECHNOLOGICAL FIELD

The present invention relates to a heart inner wall checking tool and a heart inner wall checking device which are inserted into the heart in order to check the presence or absence of thrombus formation in the heart inner wall (endocardium) when an artificial heart (in particular, a ventricular assist device, abbreviated as VAD) is connected to the heart.

### BACKGROUND ART

While the life span of humans has been lengthened due to the advancement in medical treatments, the ratio of heart failure has been on an increase. The ventricular assist device has been utilized as a replacement for cardiac functions during the period in which such cardiac functions that have been lost due to heart disease, trauma, or heart attack are in the process of recovery, during the waiting period for a heart transplant, or on a permanent basis.

Furthermore, in order for the ventricular assist device to be applied to a patient with an implanted artificial heart, the left heart wall in the case of a left ventricular assist device (LVAD), and the right heart wall in the case of a right ventricular assist device (RVAD), are incised. Subsequently, a port of the artificial heart or a tube connected to the artificial heart is inserted into the incision. This is how the artificial heart becomes connected to the heart. This surgery is normally conducted with the blood circulation of the heart stopped by utilizing an artificial cardiopulmonary device.

When the artificial heart is connected to the heart, it is necessary to check the presence or absence of thrombus formation on the surface of the heart inner wall (specifically, the endocardium). The heart with heart failure does not operate well, which causes thrombi to be formed on the endocardium. The thrombi formed on the endocardial surface might be peeled off from the endocardium and move in the blood vessels, which might contribute to the development of cerebral infarction or other conditions, due to the stimuli resulting from the procedure for connecting the ventricular assist device to the heart, or due to the improved blood flow after the artificial heart is connected. If the thrombus formation on the endocardial surface is not found, the procedure for connecting the artificial heart is continued. If the thrombus formation on the endocardial surface is found, the thrombi are carefully removed through the utilization of a pair of tweezers under direct observation. In this procedure, the heart of the patient is stopped, and so-called extracorporeal blood circulation is carried out, in which an externally installed artificial cardiopulmonary device is connected to the heart. The stoppage of the heart and the extracorporeal blood circulation impose a heavy load on the patient, and the patient requires an extended amount of time to recover from such surgery.

In recent years, in order to solve the above-stated problem, a procedure in which the surgery is conducted without stopping the heart (off-pump surgery) has been conducted in bypass surgeries, etc. Even in the procedures for installing the artificial heart, a technique that does not involve the stoppage of the heart has been attempted. However, some patients with heart failure have already developed thrombi on the heart walls due to the slow movements of the heart walls. The thrombi might move to other locations after the installation, which might develop cerebral infarction and other conditions. That is why the use of the procedure is not very commonly conducted. In the currently-available checking methods such as ultrasound imaging and the like, it is difficult to externally check the presence of a thrombus layer on the heart walls. The difficulty of checking the presence of the thrombus layer has been a barrier to the off-pump surgery in the procedure for installing the artificial heart.

### DISCLOSURE OF THE INVENTION

### Problem that the Invention is to Solve

The purpose of the present invention is to provide a heart inner wall checking tool and a heart inner wall checking device which are capable of easily checking the presence or absence of thrombus formation on the heart inner wall (the surface of the endocardium) through the utilization of the incision formed on the location of the heart to which the artificial heart is connected in the procedure for connecting the artificial heart to the heart.

### Means for Solving the Problem

The above-stated purpose is achieved by the following:

A heart inner wall checking tool comprising a tubular main body and an inflatable balloon provided on the distal end of the tubular main body;
wherein the heart inner wall checking tool comprises a liquid injection port for injecting a inflating liquid into the balloon and an imaging member port for inserting a shaft-shaped imaging member, the balloon possesses transparency which enables imaging of the outside of the balloon through the use of the shaft-shaped imaging member, the balloon has a balloon rear portion which is capable of coming into close contact with the heart inner wall when the balloon is inflated, and the balloon rear portion comprises a liquid-exudable member which exudes the injected inflating liquid and supplies the inflating liquid between the balloon rear portion and the heart inner wall.

The above-stated purpose is also achieved by the following: A heart inner wall checking device comprising the heart inner wall checking tool, and a shaft-shaped imaging member which can be inserted into the heart inner wall checking tool or minimally the distal end of which is accommodated within the heart inner wall checking tool; wherein the shaft-shaped imaging member is capable of photographing the heart inner wall from the inner rear side of the inflated balloon.

### BRIEF EXPLANATION OF THE DRAWINGS

FIG. 1 is an external view of the heart inner wall checking tool of the present invention in a working example.

FIG. 2 is a longitudinal sectional view of the heart inner wall checking tool shown in FIG. 1.

FIG. 3 is an external view of the heart inner wall checking tool shown in FIG. 1 when the balloon is inflated.

FIG. 4 is a longitudinal sectional view of the heart inner wall checking tool in the status shown in FIG. 3.

FIG. 5 is an enlarged sectional view showing the distal end of the heart inner wall checking tool shown in FIG. 4.

FIG. 6 is a sectional view taken along line A-A of the heart inner wall checking tool shown in FIG. 5.

FIG. 7 is an external view of the heart inner wall checking tool of the present invention in another working example.

FIG. 8 is a longitudinal sectional view of the heart inner wall checking tool shown in FIG. 7.

FIG. 9 is an explanatory diagram to explain the operations of the heart inner wall checking tool shown in FIG. 7 and FIG. 8.

FIG. 10 is an enlarged view showing the distal end of the heart inner wall checking tool of the present invention in yet another working example.

FIG. 11 is a sectional view taken along line B-B of the heart inner wall checking tool shown in FIG. 10.

FIG. 12 is an enlarged view showing the distal end of the heart inner wall checking tool of the present invention in yet another working example.

FIG. 13 is a sectional view taken along line C-C of the heart inner wall checking tool shown in FIG. 12.

FIG. 14 is an enlarged view showing the distal end of the heart inner wall checking tool of the present invention in yet another working example.

FIG. 15 is a sectional view taken along line D-D of the heart inner wall checking tool shown in FIG. 14.

FIG. 16 is an external view of a heart inner wall checking device of the present invention in a working example.

FIG. 17 is a longitudinal sectional view of the heart inner wall checking device shown in FIG. 16.

FIG. 18 is an enlarged view showing a shaft-shaped imaging member utilized in the heart inner wall checking device shown in FIG. 16 and FIG. 17.

FIG. 19 is an enlarged sectional view showing the shaft-shaped imaging member utilized in the heart inner wall checking device of the present invention in another working example.

FIG. 20 is an external view of the heart inner wall checking device of the present invention in another working example.

FIG. 21 is a longitudinal sectional view of the heart inner wall checking device shown in FIG. 20.

FIG. 22 is an explanatory diagram to explain the operations of the heart inner wall checking tool and the heart inner wall checking device of the present invention.

### BEST MODE FOR CARRYING OUT THE INVENTION

An explanation regarding the heart inner wall checking tool and the heart inner wall checking device in the working examples shown in the drawings is provided below. A heart inner wall checking device 100 comprises heart inner wall checking tool 1 and shaft-shaped imaging member 5, which can be inserted into heart inner wall checking tool 1 or minimally the distal end of which is accommodated within the heart inner wall checking tool.

Heart inner wall checking tool 1 of the present invention comprises tubular main body 2, and inflatable balloon 3, which is provided on a distal end of tubular main body 2. Heart inner wall checking tool 1 also comprises liquid injection port 42 for injecting an inflation liquid into balloon 3, and imaging member port 43 for inserting shaft-shaped imaging member 5. Balloon 3 possesses transparency, which enables imaging of the outside of balloon 3 through the use of area image sensor 51, and includes balloon rear portion [TN: also referred to as “rear protruding member”] 32, which is capable of coming into close contact with the heart inner wall when balloon 3 is inflated. Balloon rear portion 32 comprises liquid-exudable member 33, which exudes the injected inflating liquid and supplies the inflating liquid between balloon rear portion 32 and heart inner wall 12.

Heart inner wall checking tool 1 of the working examples shown in the drawings comprises tubular main body 2, inflatable balloon 3, which is provided on the distal end of tabular main body 2, and hub 4, which is provided on the proximal end of tubular main body 2.

Tubular main body 2 is a tubular body which possesses inner space 20 continuous from the distal end to the proximal end thereof. Moreover, on the distal end of tubular main body 2, balloon fixating member 21 is provided; and on the proximal end, hub mounting member 22 is provided.

Moreover, in this working example, the proximal end of tubular main body 2 has an enlarged diameter. In addition, inner space 20 of tubular main body 2 functions as an insertion space for shaft-shaped imaging member 5 as well as a pathway for the inflating liquid.

Tubular main body 2 preferably possesses transparency which enables viewing the inside thereof. Favorable examples of the material with which tubular main body 2 may be constituted include hard or semi-hard synthetic resins, such as polycarbonate, acrylic resins (polyacrylate, polyacrylamide, polyacrylonitrile-styrene copolymers, acrylonitrile-butadiene-styrene copolymers, etc.), polyesters (polyethylene terephthalate, polybutylene terephthalate), polyolefins (polyethylene, polypropylene, ethylene-propylene copolymers), styrene-based resins (polystyrene), MS resins (methacrylate-styrene copolymers), and MBS resins (methacrylate-butylene-styrene copolymers) [parenthesis mistake in the source; best translation rendered]. Moreover, tubular main body 2 may be formed with a metal tube (for example, a stainless tube).

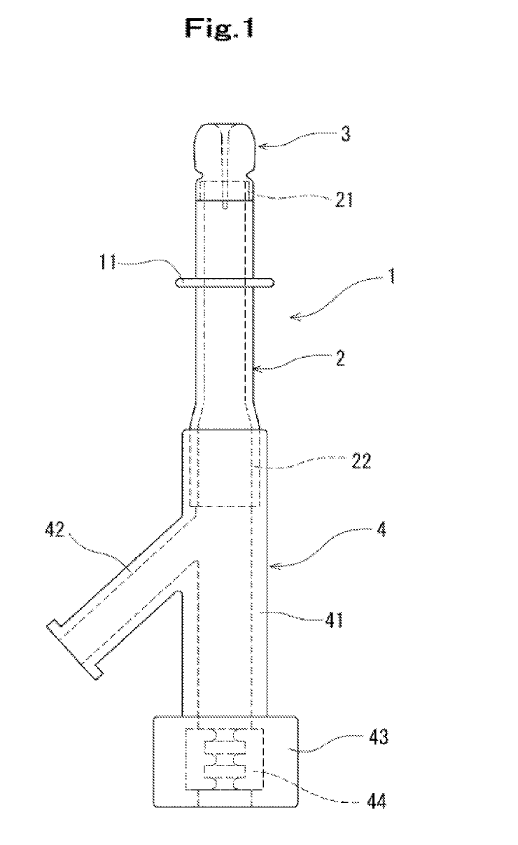

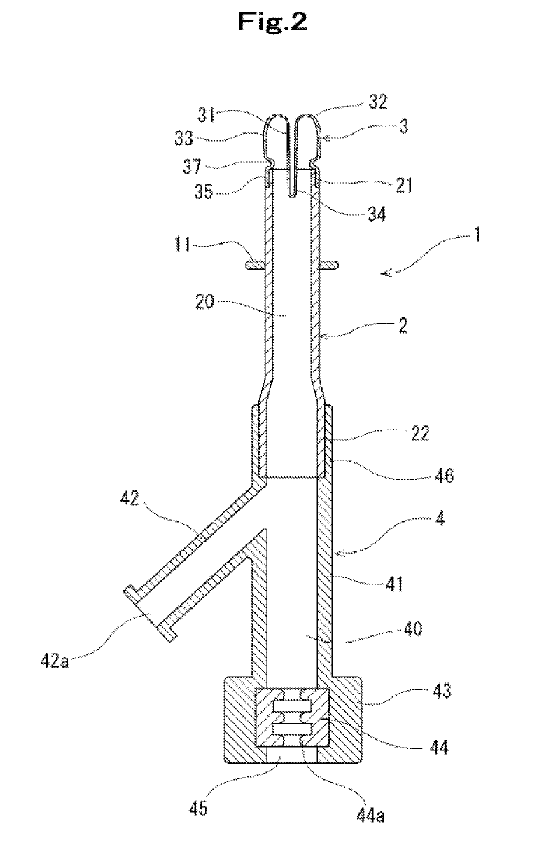

Heart inner wall checking tool 1 of this working example further comprises a fixating member 11, which fixates heart inner wall checking tool 1 to the heart. Fixating member 11, as shown in FIG. 1 and FIG. 2, is preferably a fixating annular member which is mounted in a movable manner on the outer surface of tubular main body 2. In addition, the fixating annular member may take a short cylindrical shape.

Balloon 3 is fixated onto the distal end of tubular main body 2. Balloon 3 is inflated (expanded) into a certain shape by the injected inflating liquid. In addition, balloon 3 possesses transparency which enables imaging of the outside of the inflated member through the use of the area image sensor when it is inflated by the inflating liquid. Balloon 3 has an approximate perfect circle-shaped opening on the rear edge of the central portion thereof; and the opening is fixated onto balloon fixating member 21 of tubular main body 2. As the inflating liquid flows into the blood stream through micropores, physiological saline solution is preferably utilized as the inflating liquid.

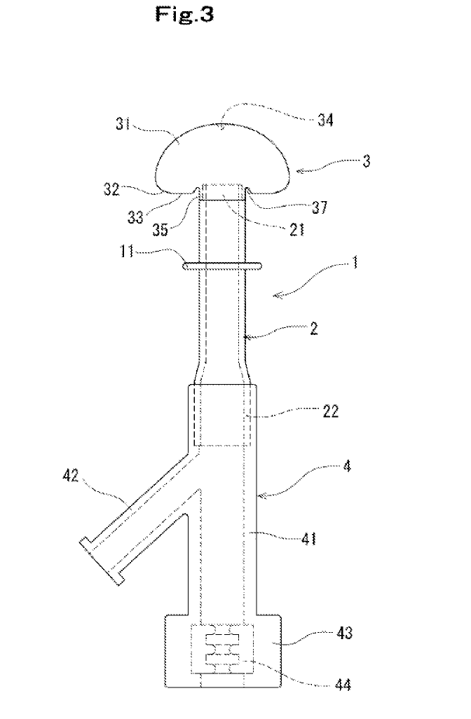

Balloon 3 can be squashed or miniaturized, and it has inner space 30, as shown in FIG. 2; when the inflating liquid flows into it, balloon 3 can be inflated into a mushroom-like shape as shown in FIG. 3 to FIG. 5.

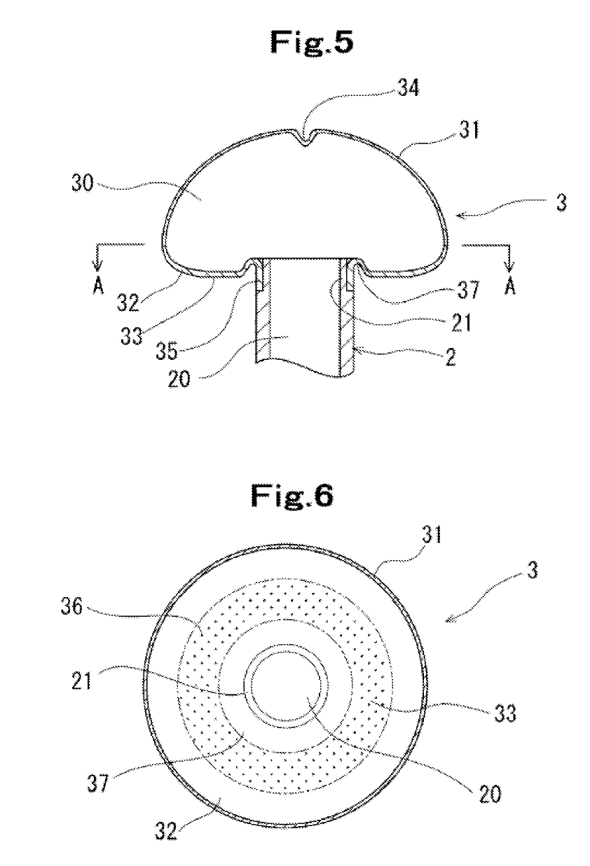

Balloon 3 comprises balloon main body 31 and opening 35, which is fixated onto fixating member 21 of tubular main body 2. Balloon 3 further comprises balloon rear portion 32, which is capable of coming into close contact with the heart inner wall when balloon 3 is inflated. Balloon rear portion 32 comprises liquid-exudable member 33, which exudes the injected inflating liquid and supplies the inflating liquid between balloon rear portion 32 and heart inner wall 12. In particular, as shown in FIG. 5 and FIG. 6, in balloon 3 in this working example, balloon rear portion 32 is intended to protrude in the rear of junction upper end 37 provided between balloon 3 and tubular main body 2 when balloon 3 is inflated. In other words, when balloon 3 is inflated, balloon rear portion 32 thereof is designed to protrude in the rear direction from the distal end of tubular main body 2.

Balloon rear portion 32 has liquid-exudable member 33. Liquid-exudable member 33 is formed in a ring-shaped area having a certain width so as to surround the peripheral edge of the opening of balloon 3 (namely, the distal end of the tubular main body). In addition, as shown in FIG. 5 and FIG. 6, balloon rear portion 32 possesses a portion which becomes flat when the balloon is inflated; and on this flat portion, liquid-exudable member 33 is formed. The width of the ring-shaped area on which liquid-exudable member 33 is formed is preferably in the range from 5 mm to 2.0 mm, and more preferably in the range from 7 mm to 10 mm. The outer diameter of the ring-shaped area is preferably in the range from 15 mm to 30 mm, and more preferably in the range from 17 mm to 25 mm. The inner diameter of the ring-shaped area is preferably in the range from 9 mm to 25 mm, and more preferably in the range from 12 mm to 17 mm. As for the size of balloon 3, the outer diameter at the portion where the outer diameter of balloon rear portion 32 becomes the largest when the balloon is inflated is preferably in the range from 20 mm to 35 mm, and more preferably in the range from 22 mm to 30 mm. Liquid-exudable member 33 has a numerous number of micropores 36, through which the inflating liquid is exuded into balloon 3. The diameter of a micropore is preferably in the range from 100 μm to 1000 μm, and more preferably in the range from 300 μm to 700 μm. The number of the micropores (micropore density) is preferably in the range from 0.1 micropore/mm² to 2 micropore/mm², and more preferably in the range from 0.7 micropore/mm² to 1.2 micropore/mm². Moreover, in balloon 3 of this working example, junction upper end 37 is an annular curved portion which is shaped so as to be radically curved in the rear direction; and on this portion, liquid-exudable member 33 is not provided. Balloon 3 further comprises a distal end recess 34, which is formed in order to render pressing down the balloon easier.

As for the material to form balloon 3, materials with a certain degree of flexibility may be favorably utilized. Examples of such materials include thermoplastic resins such as polyolefins (e.g.: polyethylene, polypropylene, ethylene-propylene copolymers, ethylene-vinyl acetate copolymers, crosslinked ethylene-vinyl acetate copolymers, etc.), polyvinyl chloride, polyamide elastomer, polyurethane, polyester (e.g.: polyethylene terephthalate), polyarylene sulfide (e.g.: polyphenylene sulfide), and the like; silicone rubber, latex rubber, and the like. In particular, stretchable materials are preferable, and balloon 3 is preferably formed with the materials possessing a high degree of transparency and a certain degree of strength. Moreover, balloon 3 is preferably colorless and transparent. In addition, balloon 3 may be formed to be integrated with tubular main body 2.

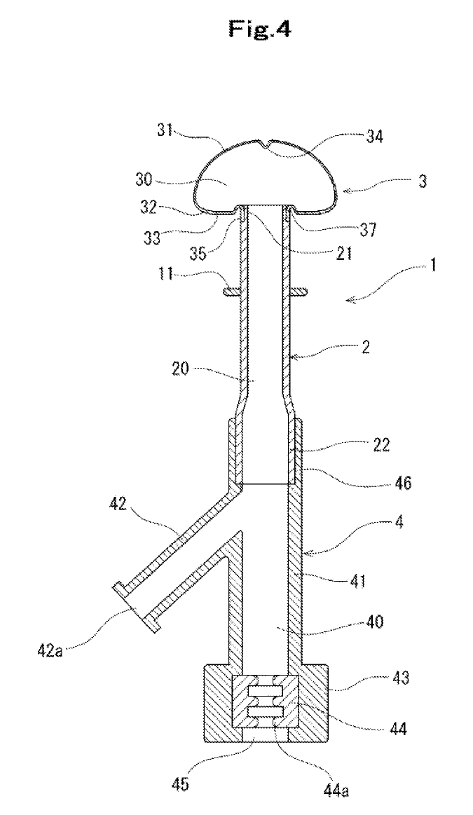

Moreover, as shown in FIG. 1 through FIG. 4, heart inner wall checking tool 1 comprises hub 4; and hub 4 may be formed to be integrated with tubular main body 2. In this working example, the distal end of hub 4 is fixated at the proximal end of tubular main body 2 for fixating the hub (hub mounting member 22). Moreover, in this working example, hub 4 comprises hub main body 41; liquid injection port 42, which extends from a side of hub main body 41 in a branched manner; imaging member port 43, which is to insert shaft-shaped imaging member 5; and distal end 46, which is to be joined with hub mounting member 22 of tubular main body 2. Hub 4 is a so-called branch hub. Liquid injection port 42 comprises a lumen which is connected to inner space 20 of tubular main body 2; and similarly as liquid injection port 42, imaging member port 43 also comprises lumen 40, which is connected to inner space 20 of tubular main body 2, as well as port opening 45. Furthermore, in this working example, as shown in FIG. 2 and FIG. 4, sealing member 44, which renders shaft-shaped imaging member 5 to be slidable and rotatable under liquid tightness condition, is provided within hub 4. Sealing member 44 comprises ring-shaped protruding member 44a, which comes in contact with the outer surface of shaft-shaped main body 53 of shaft-shaped imaging member 5 under the condition of liquid tightness. In addition, ring-shaped protruding member 44a allows the shift of shaft-shaped imaging member 5 to move in the axial direction with the liquid tightness status maintained.

In particular, in the hub of this working example, sealing member 44 is accommodated within the rear end of hub 4, and possesses a multiple number of ring-shaped protruding members 44a. Moreover, liquid injection port 42 comprises opening 42a, to which a liquid injector (for example, a syringe) can be connected.

As the materials to form hub 4, the materials listed as the materials to form tubular main body 2 may be favorably utilized. Moreover, as the materials to form sealing member 44, flexible materials are utilized. Examples of such flexible materials include rubbers such as synthetic rubbers (e.g.: urethane rubber, silicone rubber, butadiene rubber, etc.) and natural rubbers (e.g,: latex rubber, etc.); and synthetic resin elastomers such as olefin-based elastomers (e.g.: polyethylene elastomers, polypropylene elastomers), polyamide elastomers, styrene-based elastomers (e.g.: styrene-butadiene-styrene copolymers, styrene-isoprene-styrene copolymers, styrene-ethylene butylate-styrene copolymers), polyurethane, urethane-based elastomers, fluorine resin-based elastomers, and the like.

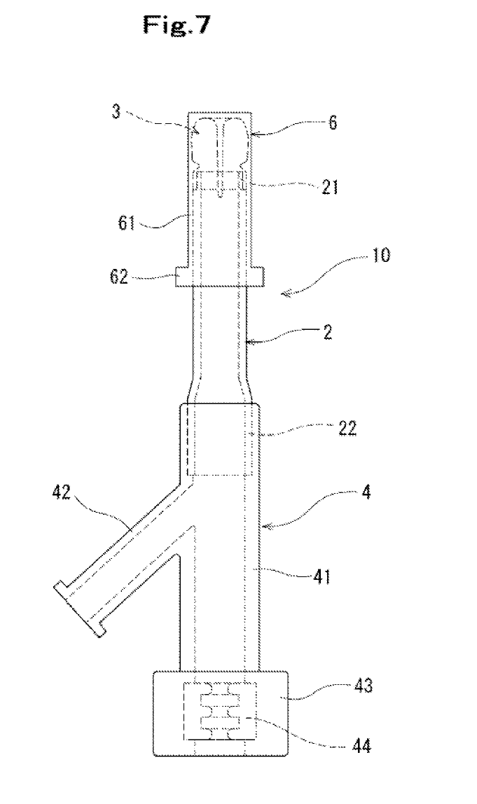

Subsequently, an explanation regarding heart inner wall checking device 10 of the present invention in another working example shown in FIG. 7 through FIG. 9 is provided below.

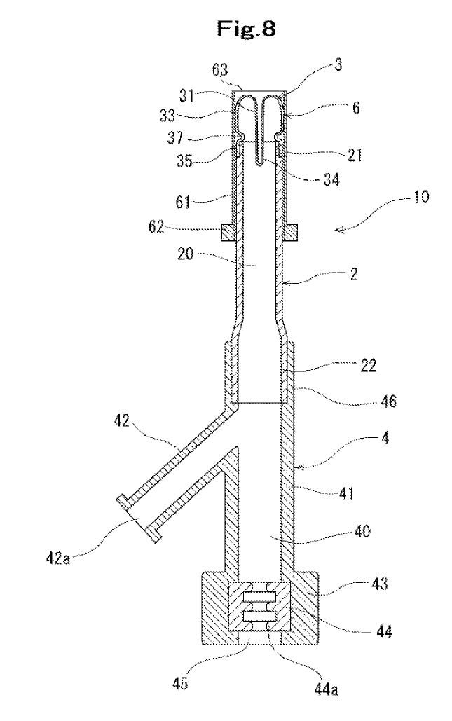

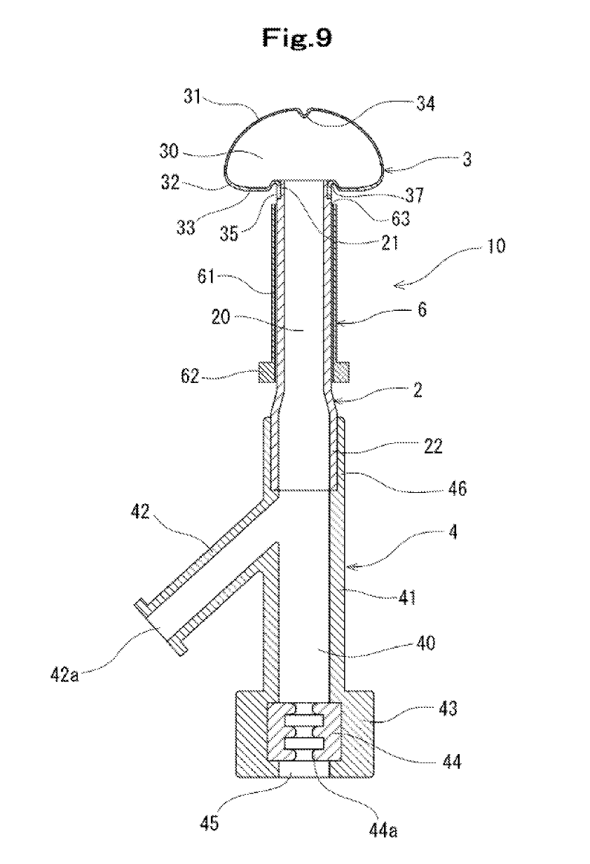

FIG. 7 is an external view of the heart inner wall checking tool of the present invention in another working example. FIG. 8 is a longitudinal sectional view of the heart inner wall checking tool shown in FIG. 7. FIG. 9 is an explanatory diagram to explain the operations of the heart inner wall checking tool shown in FIG. 7 and FIG. 8.

The only difference between heart inner wall checking device 10 and heart inner wall checking tool 1 is the presence or absence of sheath 6 and fixating member 11. The same reference numerals are given to the same members, and the explanation above is omitted.

Heart inner wall checking device 10 in this working example, as shown in FIG. 7 through FIG. 9, comprises sheath 6, which accommodates balloon 3 and the distal end of tubular main body 2; but heart inner wall checking device 6 does not include fixating member 11.

Sheath 6, as shown in FIG. 7 through FIG. 9, is a cylindrical tube which possesses inner space continuous from one end to the other end. Sheath 6 comprises main body 61, operating grip member 62 provided at the proximal end, and distal end opening 63.

Moreover, as shown in FIG. 8, balloon 3 and the distal end of tubular main body 2 are accommodated within sheath 6. Due to the constitution stated above, in heart inner wall checking device 10, the distal end is formed with the tube-shaped sheath member, which renders the insertion of the device into the heart easier. Moreover, the inner surface of sheath 6 is in contact with the outer surface of balloon 3 as well as the outer surface of tubular main body 2. Thus, unless sheath 6 is manipulated, balloon 3 is not exposed. As for the materials to form sheath 6, the materials listed as the materials to form tubular main body 2 may be favorably utilized.

Furthermore, the inner surface of the sheath is preferably treated with a treatment for reducing the sliding resistance. Such a treatment as the above can be achieved through methods in which hydrophilic polymer (e.g.: poly(2-hydroxyethyl methacrylate), poly-hydroxyethyl acrylate, hydroxypropyl cellulose, methyl vinyl ether-maleic anhydride copolymer, polyethylene glycol, polyacrylamide, polyvinylpyrrolidone, etc.) is coated or fixated.

Moreover, in heart inner wall checking device 10 of the present invention, as shown in FIG. 9, when sheath 6 is shifted towards the proximal end of tubular main body 2, balloon 3 becomes exposed and ready to be inflated. Balloon 3 is inflated by the injection of liquid.

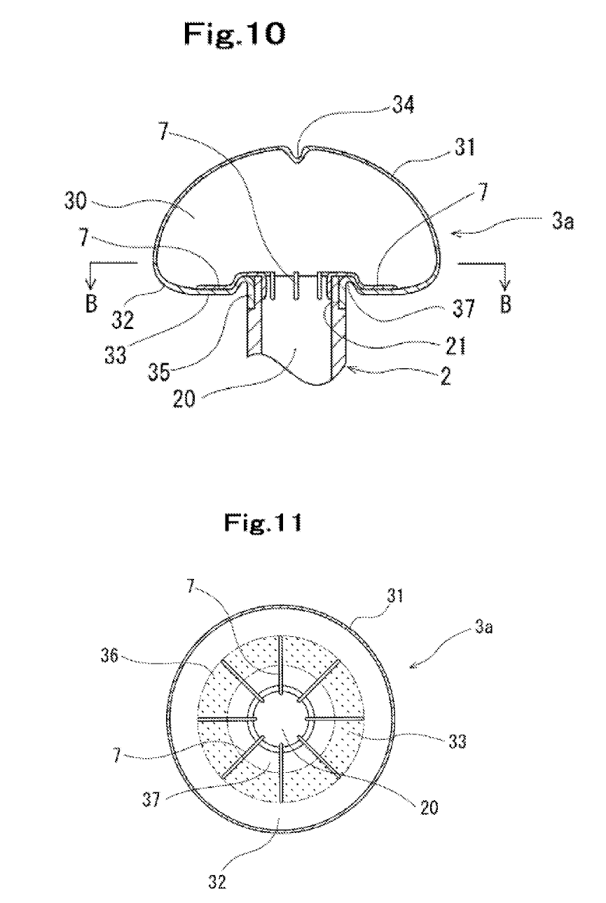

The balloon utilized in the heart inner wall checking tool of the present invention is not limited to balloon 3 stated above. For example, the balloon may be balloon 3a shown in FIG. 10 and FIG. 11. Balloon 3a comprises rear-protruding-member forming member 7 for forming rear protruding member 32 which protrudes in the rear of the junction upper end provided between balloon 3a and tubular main body 2 at the time of balloon 3a being inflated. Rear-protruding-member forming member 7 is designed as follows: one end thereof is fixated onto the distal end of tubular main body 2 and goes into the balloon; and the other end thereof reaches the outer edge of the ring-shaped area where liquid-exudable member 33 is formed. Moreover, a multiple number of rear-protruding-member forming members 7 are provided; these extend from the distal end opening of tubular main body 2 in a radial manner. Furthermore, multiple rear-protruding-member forming members 7 are arranged so that each member achieves an equal angle with respect to the central axis of the tubular main body. In addition, rear-protruding-member forming members 7 are formed with a flexible material which has been shaped into the shape shown in FIG. 10.

Due to the above, [rear-protruding-member forming members 7] are securely inflated into the shape shown in FIG. 10 when inflated. Rear-protruding-member forming members 7 preferably retain the flexibility enabling pressing down the balloon as well as the shape-retaining capability for the above-stated shape. Examples of the material to form rear-protruding-member forming members 7 include polyesters (polyethylene terephthalate, polybutylene terephthalate), polyolefins (polyethylene, polypropylene, ethylene-propylene copolymers), polyamides, and the like.

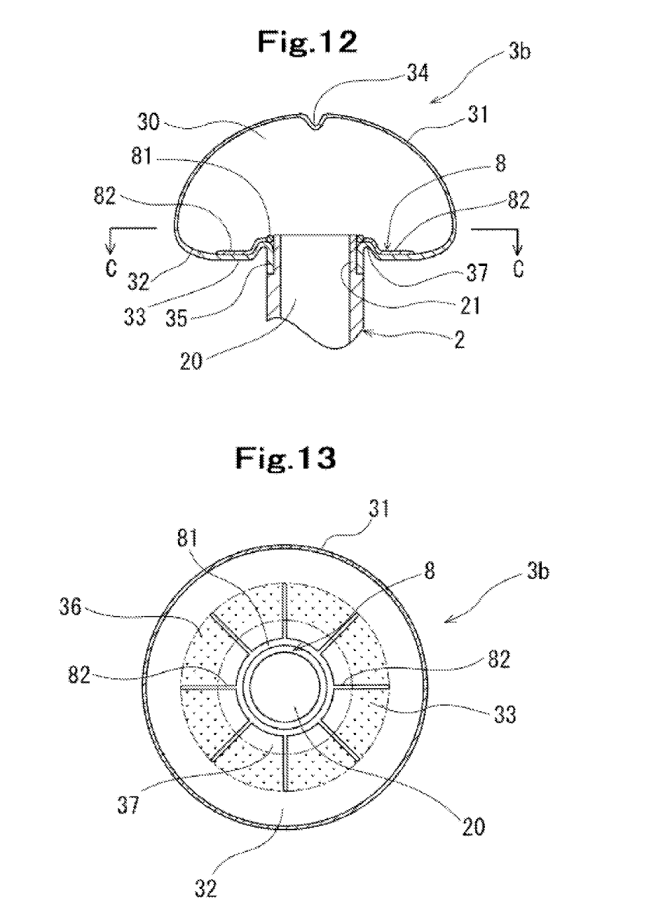

The balloon may also be balloon 3b shown in FIG. 12 and FIG. 13. Balloon 3b comprises a rear-protruding-member forming member, similarly to balloon 3a stated above. Rear-protruding-member forming member 8 comprises ring-shaped member 81, which is fixated onto tubular main body 2; as well as a multiple number of linear members 82, of which one end is in ring-shaped member 81 and the other end reaches liquid-exudable member 33. Specifically, ring-shaped member 81 of rear-protruding-member forming member 8 is fixated onto the distal end outer surface of tubular main body 2 and the upper edge surface of balloon 3b. Linear member 82 is designed as follows: one end thereof is in ring-shaped member 81 and goes into the balloon, and the other end thereof reaches the outer edge of the ring-shaped area where liquid-exudable member 33 is formed. A multiple number of linear members 82 are provided, and extend from the distal end opening of tubular main body 2 in a radial manner. Furthermore, multiple linear members 82 are arranged so that each member achieves an equal angle with respect to the central axis of the tubular main body. Moreover, rear-protruding-member forming member 8 is formed with a flexible material which has been shaped into the shape shown in FIG. 12. Due to the above, [rear-protruding-member forming members 8] are securely inflated into the shape shown in FIG. 12 when inflated. Rear-protruding-member forming members 8 preferably retain the flexibility that enables pressing down of the balloon as well as the shape-retaining capability for the above-stated shape. As for the material to form rear-protruding-member forming members 8, the materials listed as the materials to form rear-protruding-member forming members 7 may be favorably utilized.

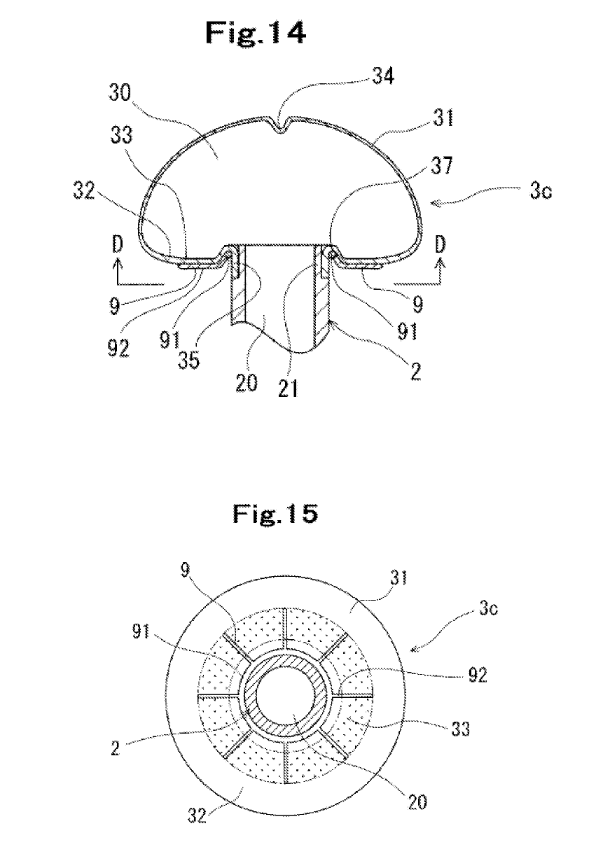

The balloon may also be balloon 3c shown in FIG. 14 and FIG. 15. Balloon 3c comprises a rear-protruding-member forming member, similarly to balloon 3a stated above. Rear-protruding-member forming member 9 comprises ring-shaped member 91, which is fixated onto the outer surface of junction upper end 37 joining balloon 3c and the distal end of tubular main body 2; as well as a multiple number of linear members 92, of which one end is in ring-shaped member 91 and the other end reaches liquid-exudable member 33.

Specifically, ring-shaped member 91 of rear-protruding-member forming member 9 is fixated onto the outer surface of balloon 3c. Linear member 92 is designed as follows: one end thereof is in ring-shaped member 91 and extends on the outer surface of balloon 3c; and the other end thereof reaches the outer edge of the ring-shaped area where liquid-exudable member 33 is formed. A multiple number of linear members 92 are provided, and extend in a radial manner. Furthermore, multiple linear members 92 are arranged so that each member achieves an equal angle with respect to the central axis of the tubular main body. Moreover, rear-protruding-member forming member 9 is formed with a flexible material which has been shaped into the shape shown in FIG. 14. Due to the above, [rear-protruding-member forming members 9] are securely inflated into the shape shown in FIG. 14 when inflated. Rear-protruding-member forming members 9 preferably retain the flexibility enabling pressing down the balloon as well as the shape-retaining capability for the above-stated shape. As for the material to form rear-protruding-member forming members 9, the materials listed as the materials to form rear-protruding-member forming members 7 may be favorably utilized.

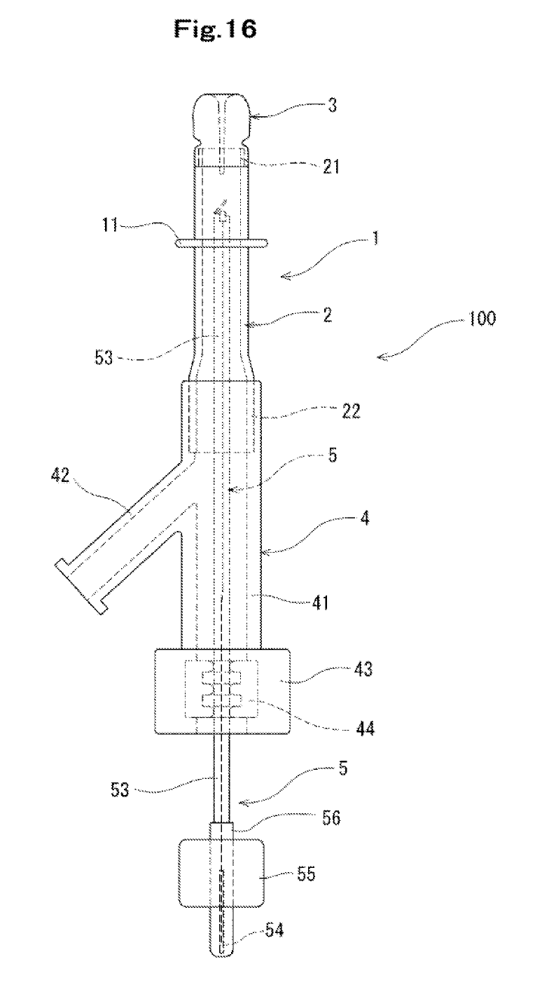

An explanation regarding heart inner wall checking device 100 of the present invention shown in FIG. 16 through FIG. 18 is provided below.

Heart inner wall checking device 100 comprises the above-stated heart inner wall checking tool 1 and shaft-shaped imaging member 5, which can be inserted into heart inner wall checking tool 1. As the heart inner wall checking tool, all of the heart inner wall checking tools in all the working examples stated above may be utilized. In particular, heart inner wall checking device 100 shown in FIG. 16 through FIG. 18 utilizes heart inner wall checking tool 1 stated above.

In heart inner wall checking device 100, shaft-shaped imaging member 5 is removable from heart inner wall checking tool 1. In other words, shaft-shaped imaging member 5 can be inserted with its distal end first through imaging member port 43 of heart inner wall checking tool 1. Moreover, shaft-shaped imaging member 5 may be designed in a manner so that at least the distal end thereof is accommodated within heart inner wall checking tool 1. Furthermore, as stated above, hub 4 of heart inner wall checking tool 1 comprises sealing member 44 which renders shaft-shaped imaging member 5 to be slidable and rotatable under liquid tightness condition.

For shaft-shaped imaging member 5, an imaging tool which has its range of vision on the proximal end side and is capable of imaging the rear area is utilized, and the imaging member is capable of imaging the heart inner wall from the inner rear portion of inflated balloon 3.

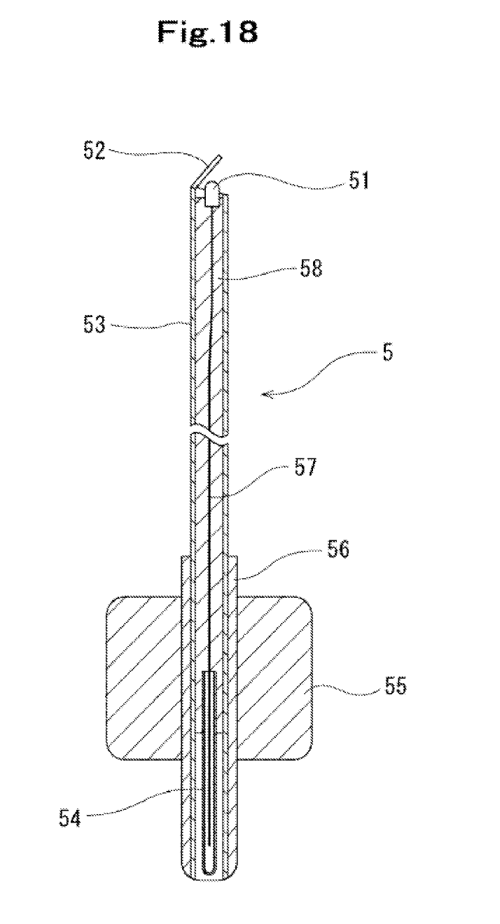

Shaft-shaped imaging member 5 shown in FIG. 18 comprises imaging element 51 (specifically, an area image sensor) on the distal end thereof, and thus the imaging member is capable of checking the rear area. Shaft-shaped imaging member 5 in this working example comprises shaft-shaped main body 53; area image sensor 51, which is provided on the distal end of shaft-shaped main body 53; and connector 56, which is provided on the proximal end of shaft-shaped main body 53 and possesses contact point 54 electrically connected to area image sensor 51. Moreover, in shaft-shaped imaging member 5 in this working example, shaft-shaped imaging member 5 comprises area image sensor 51, which is provided so as to face forward, and mirror 52, which enables area image sensor 51 to image the targets in the diagonal rear direction.

The area image sensor is a type of solid imaging elements; the CCD (Charge Coupled Device) image sensor and CMOS (Complementary Metal Oxide Semiconductor) image sensor are preferable. Moreover, for the area image sensor utilized in the present invention, image sensors which are capable of wide-angle imaging are particularly preferable. Furthermore, the area image sensor is preferably equipped with a lighting function. A preferable lighting function is Light Emitting Diode (not shown in the figures). Shaft-shaped imaging member 5 comprises connector 56, which is provided on the rear end of shaft-shaped main body 53; and connector 56 further comprises contact point 54, which is electrically connected to area image sensor 51 through cord 57. To connector 56, an image outputting device (not shown in the figures) is connected when the tool is in use. Moreover, on the proximal end of shaft-shaped imaging member 5, operating member 55 is provided. Within shaft-shaped main body 53, filler 58 is injected.

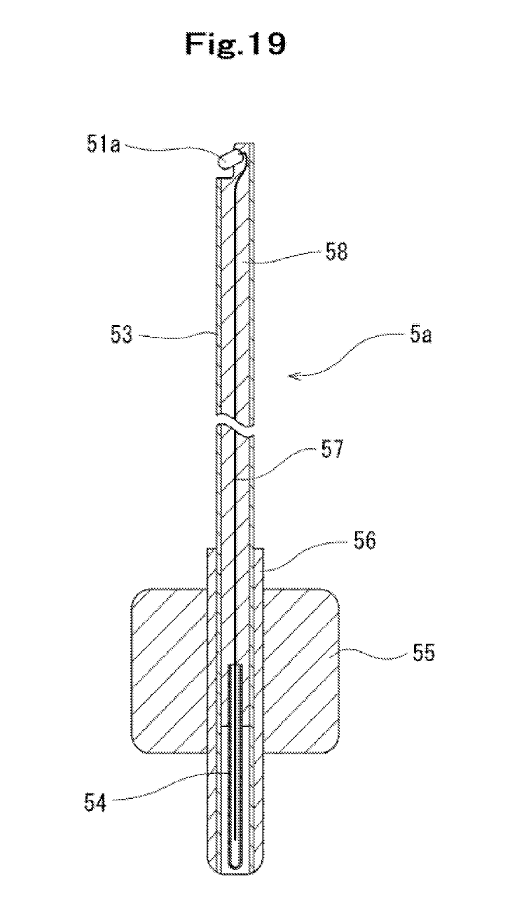

The shaft-shaped imaging member is not limited to the ones stated above. For example, it may be shaft-shaped imaging member 5a shown in FIG. 19. In shaft-shaped imaging member 5a of this working example, area image sensor 51a is provided on the distal end of shaft-shaped main body 53 so as to face in the diagonal rear direction, and it is not equipped with any mirror. Other aspects of this imaging member is the same as shaft-shaped imaging member 5 stated above.

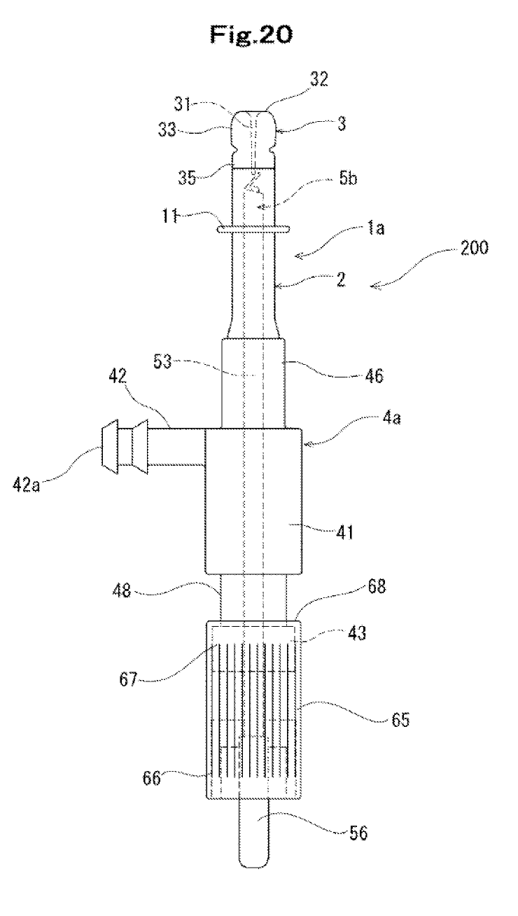

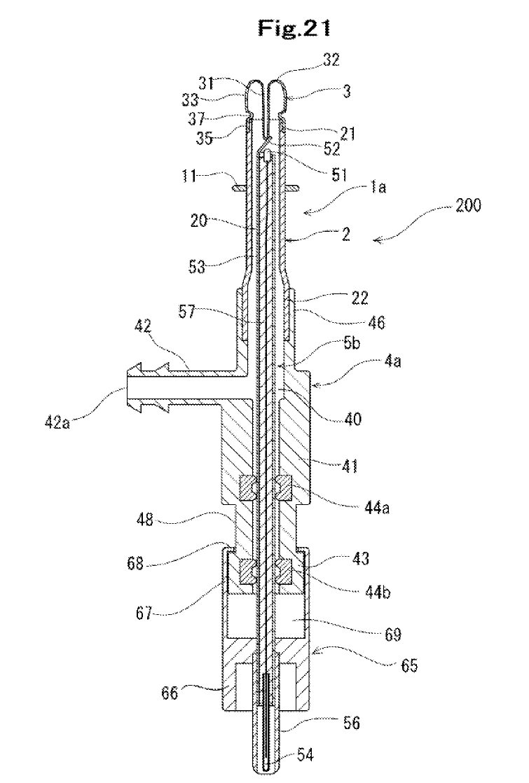

An explanation regarding heart inner wall checking device 200 of the present invention shown in FIG. 20 and FIG. 21 is provided below.

Heart inner wall checking device 200 comprises a heart inner wall checking tool 1a and a shaft-shaped imaging member 5b. Moreover, as the shaft-shaped imaging member, area image sensor 51a, which functions similarly to above-stated shaft-shaped imaging member 5a, is provided on the distal end of shaft-shaped main body 53 so as to face in the diagonal rear direction, and it may not be equipped with any mirror.

Moreover, in this working example, shaft-shaped imaging member 5b, excluding the proximal end thereof, is accommodated within heart inner wall checking tool 1a. Moreover, operating grip member 65 of shaft-shaped imaging member 5b is equipped with engaging member 68, which is provided on the rear end of huh 4a of heart inner wall checking tool 1a in a non-removable manner and in a manner movable in a certain longitudinal direction.

In heart inner wall checking tool 1a utilized in heart inner wall checking device 200 in this working example, the shape of the hub is different from that of heart inner wall checking tool 1. The remaining members (balloon 3, tubular main body 2, fixating member 11, etc.) are the same; these members are referred to by the same reference numerals, and the explanation on these members is omitted.

Huh 4a of heart inner wall checking tool 1a utilized in this working example comprises hub main body 41; liquid injection port 42, which extends so as to be orthogonal to the central axis thereof; and junction member 46 for joining with the tubular main body. Moreover, to liquid injection port 42, the injection tube of the liquid supplier can be connected. Moreover, hub 4a comprises smaller-diameter member 48, which extends from hub main body 41 towards the rear end; and proximal-end member 43, which extends from smaller-diameter member 48 towards the rear end and has a larger diameter than the smaller-diameter member. Moreover, within hub 4a, two sealing members 44a and 44b are accommodated. The sealing members are in contact with the outer surface of shaft-shaped imaging member 5b under a liquid tightness condition, and makes the sliding and rotating possible under the liquid tightness condition.

Shaft-shaped imaging member 5b comprises operating grip member 65. Operating grip member 65 further comprises retainer 66, which retains the shaft-shaped main body and connector 56; hollow member 67, which extends from retainer 66 towards the distal end; and engaging member 68, which is formed by a ring-shaped inner protruding member provided on the distal end of hollow member 67. In hollow member 68 [sic; hollow member 67], proximal-end member 43 of hub 4a is accommodated in internal space 69 thereof. In other words, hollow member 68 [sic; hollow member 67] has a larger inner diameter than the outer diameter of proximal-end member 43 of hub 4a. Moreover, the inner diameter of engaging member 68 formed by the ring-shaped inner protruding member is smaller than the outer diameter of proximal-end member 43 of hub 4a, and larger than the outer diameter of smaller-diameter member 48. Because of the constitution stated above, operating grip member 65 of shaft-shaped imaging member 5b is rendered to be non-removable from hub 4a. Moreover, as shown in FIG. 21, operating grip member 65 of shaft-shaped imaging member 5b is designed to be transportable and rotatable in the axial direction for a predetermined distance (until engagement member 68 comes into contact with hub main body 41; or until the inner surface rear edge of hollow member 67 comes into contact with proximal-end member 43 of hub 4a) towards the distal end.

Furthermore, in the heart inner wall checking tool utilized in this working example, the balloon may be balloons 3a, 3b, and 3c, which further comprises the rear-protruding-member forming member as stated above.

Furthermore, in all the working examples stated above, the shaft-shaped imaging member is not limited to such a shaft-shaped imaging member that possesses an imaging element on the distal end thereof. For example, the shaft-shaped imaging member may be designed to comprise optical fibers equipped with a lens (an object lens) on the distal end thereof, and an imaging element provided on the proximal end of the optical fibers, wherein, similarly to the above, the heart inner wall can be imaged from the inner rear side of the inflated balloon. Even with this type of shaft-shaped imaging member, it is preferable that the shaft-shaped imaging member is equipped with a lighting function similarly to the above-stated shaft-shaped imaging members.

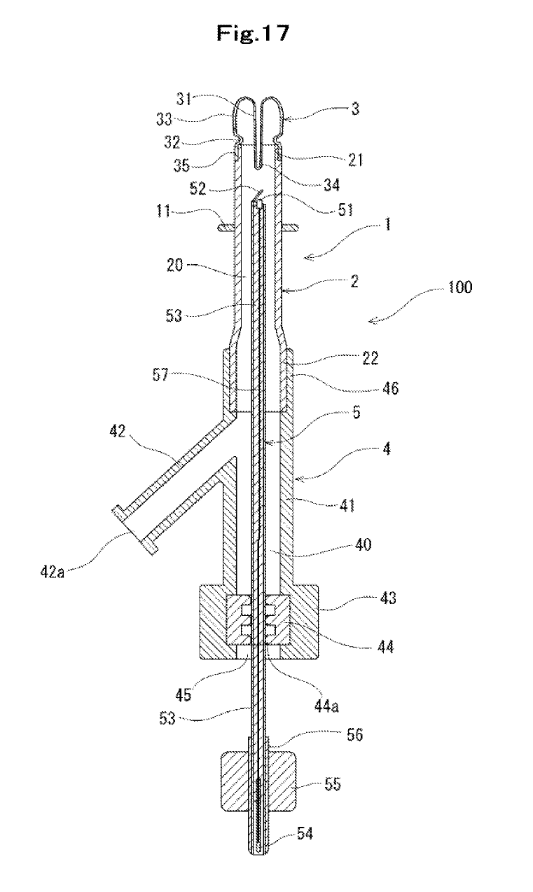

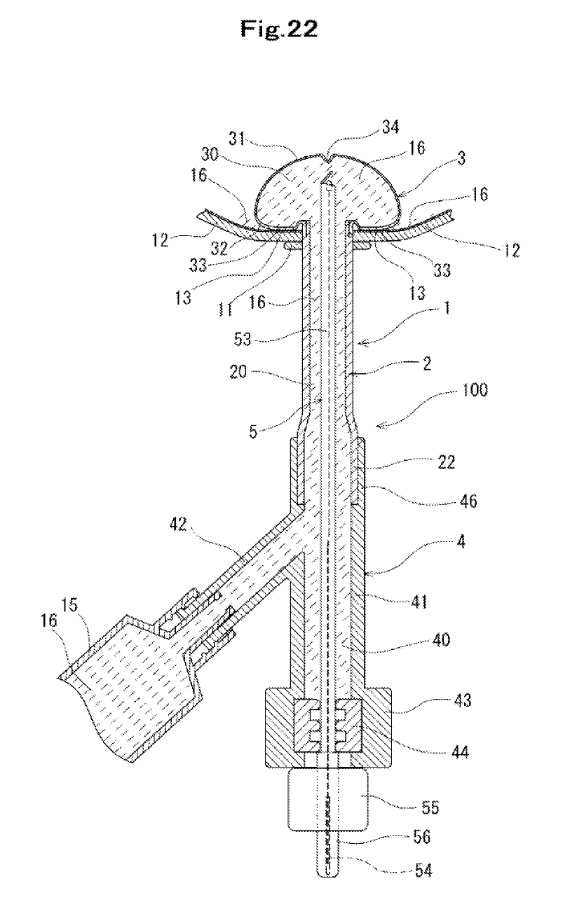

An explanation of the operations of the heart inner wall checking tool and the heart inner wall checking device of the present invention is provided below by referring to FIG. 16, FIG. 17, and FIG. 22.

First of all, as shown in FIG. 16 and FIG. 17, heart inner wall checking device 100, into which shaft-shaped imaging member 5 has been inserted in a manner so that area image sensor 51 is positioned in the internal space of the distal end of the tubular main body, is prepared within heart inner wall checking tool 1. Then liquid injector 15 (for example, a syringe), into which inflation liquid 16 has been injected, is mounted onto liquid injection port 42 of heart inner wall checking tool 1.

Furthermore, as shown in FIG. 22, the heart is partially incised, and through the incision, the above-stated heart inner wall checking device 100 is inserted into the heart in a manner so that the entirety of balloon 3 is accommodated within the heart. Subsequently, liquid injector 15 is operated to inject inflation liquid 16 through liquid injection port 42 and thereby inflate balloon 3. Then fixating member 11 is transferred towards the distal end as needed, so that the heart wall is sandwiched between inflated balloon 3 and the fixating member. Balloon 3 is inflated by injected inflation liquid 16 into a mushroom-like shape as shown in FIG. 22, which renders balloon rear portion 32 to become in contact with the heart inner wall. Furthermore, inflation liquid 16 is exuded through liquid-exudable member 33 provided on balloon rear portion 32 into between balloon rear portion 32 and heart inner wall 12. The exuded liquid pushes out the blood on the surface of endocardium 13 to form a flow of inflation liquid of a minute amount between balloon rear portion 32 and endocardium 13. Then shaft-shaped imaging member 5 is positioned in a manner so that area image sensor 51 is positioned at an inner upper position of balloon 3; and imaging of liquid-exudable member 33 is started. Images are taken with shaft-shaped imaging member 5 rotated; ring-shaped liquid-exudable member 33 is entirely imaged; and the presence or absence of thrombus on the surface of endocardium 13 is checked in the photographed images. After checking the images, inflation liquid 16 is aspirated to shrink balloon 3; and then heart inner wall checking tool 1 is removed from the heart. This completes the checking procedure.

### INDUSTRIAL APPLICABILITY

The heart inner wall checking tool of the present invention is as follows:

(1) A heart inner wall checking tool comprising a tubular main body and an inflatable balloon provided on the distal end of the tubular main body; wherein the heart inner wall checking tool comprises a liquid injection port for injecting a inflating liquid into the balloon and an imaging member port for inserting a shaft-shaped imaging member, the balloon possesses transparency which enables imaging of the outside of the balloon through the use of the shaft-shaped imaging member, the balloon has a balloon rear portion which is capable of coming into close contact with the heart inner wall when the balloon is inflated, and the balloon rear portion comprises a liquid-exudable member which exudes the injected inflating liquid and supplies the inflating liquid between the balloon rear portion and the heart inner wall.

(2) Heart inner wall checking tool of (1), wherein the shaft-shaped imaging member further comprises an imaging element on the distal end thereof, and is capable of imaging the heart inner wall from the inner rear portion of the inflated balloon.

(3) Heart inner wall checking tool of (1), wherein the shaft-shaped imaging member further comprises optical fibers equipped with a lens on the distal end thereof and an imaging element on the proximal end thereof, and is capable of imaging the heart inner wall from the inner rear portion of the inflated balloon.

(4) Heart inner wall checking tool of any of (1) to (3), wherein the balloon rear portion protrudes in the rear of a junction upper end provided between the balloon and the tubular main body when the balloon is inflated.

(5) Heart inner wall checking tool of any of (1) to (4), wherein the liquid-exudable member is formed in a ring-shaped area having a certain width so as to surround the peripheral edge of an opening of the balloon.

(6) Heart inner wall checking tool of any of (1) to (5), wherein the balloon comprises a rear-protruding-member forming member for forming the rear protruding member which protrudes in the rear of the junction upper end provided between the balloon and the tubular main body when the balloon is inflated.

(7) Heart inner wall checking tool of any of (6), wherein the rear-protruding-member forming member is formed with a multiple number of linear members, one end of which is fixated onto the tubular main body; and the other end of which reaches the liquid-exudable member.

(8) Heart inner wall checking tool of (6), wherein the rear-protruding-member forming member is formed with a ring-shaped member, which is fixated onto the tubular main body; as well as with a multiple number of linear members, one end of which is in the ring-shaped member; and the other end of which reaches the liquid-exudable member.

(9) Heart inner wall checking tool of any of (1) to (8), wherein the liquid-exudable member has a numerous number of micropores, through which the inflating liquid is exuded.

(10) Heart inner wall checking tool of any of (1) to (9), wherein the heart inner wall checking tool comprises a fixating member for fixating the heart inner wall checking tool onto the heart.

(11) Heart inner wall checking tool of any of (1) to (10), wherein the heart inner wall checking tool comprises a hub provided on the proximal end of the tubular main body, and the hub is a branch hub comprising a liquid injection port and a shaft-shaped imaging member port.

(12) Heart inner wall checking tool of any of (1) to (11), wherein the heart inner wall checking tool comprises a sheath which accommodates the balloon and the distal end of the tubular main body in a slidable manner.

(13) Heart inner wall checking tool of any of (1) to (12), wherein the heart inner wall checking tool comprises a hub which is provided on the proximal end of the tubular main body, and the hub comprises a sealing member which renders the shaft-shaped imaging member to be slidable and rotatable under liquid tightness condition.

(14) A heart inner wall checking device comprising the heart inner wall checking tool of any of (1) to (13) and a shaft-shaped imaging member which can be inserted into the heart inner wall checking tool or minimally the distal end of which is accommodated within the heart inner wall checking tool; wherein the shaft-shaped imaging member is capable of photographing the heart inner wall from the inner rear side of the inflated balloon.

(15) Heart inner wall checking device of (14), wherein the shaft-shaped imaging member comprises a shaft-shaped main body; an imaging element, which is provided on the distal end of the shaft-shaped main body; and a connector, which is electrically connected to the imaging element.

(16) Heart inner wall checking device of (15), wherein the shaft-shaped imaging member comprises the imaging element, which is provided so as to face forward, and a mirror, which enables the imaging element to image the targets in the diagonal rear direction.

(17) Heart inner wall checking device of (15), wherein the shaft-shaped imaging member comprises the imaging element, which is provided so as to face the diagonal rear direction.

(18) Heart inner wall checking device of (14), wherein the shaft-shaped imaging member comprises optical fibers equipped with a lens on the distal end thereof, and an imaging element provided on the proximal end of the optical fibers.

(19) Heart inner wall checking device of any of (14) to (18), wherein the heart inner wall checking tool comprises a hub provided on the proximal end of the tubular main body, and the hub comprises a sealing member which renders the shaft-shaped imaging member to be slidable and rotatable under liquid tightness condition.

(20) Heart inner wall checking device of any of (14) to (19), wherein the shaft-shaped imaging member can be inserted through the imaging member port.

(21) Heart inner wall checking device of any of (14) to (20), wherein the shaft-shaped imaging member comprises an operating grip member provided on the proximal end thereof.

(22) Heart inner wall checking device of (21), wherein the shaft-shaped imaging member, excluding the proximal end thereof, is accommodated within the heart inner wall checking tool, and the operating grip member of the shaft-shaped imaging member is equipped with an engaging member, which is provided on the rear end of the hub of the heart inner wall checking tool in a non-removable manner and in a manner movable in a certain longitudinal direction.

1. A heart inner wall checking tool comprising a tubular main body and an inflatable balloon provided on the distal end of the tubular main body;
wherein the heart inner wall checking tool comprises a liquid injection port for injecting a inflating liquid into the balloon and an imaging member port for inserting a shaft-shaped imaging member,
the balloon possesses transparency which enables imaging of the outside of the balloon through the use of the shaft-shaped imaging member,
the balloon has a balloon rear portion which is capable of coming into close contact with the heart inner wall when the balloon is inflated, and
the balloon rear portion comprises a liquid-exudable member which exudes the injected inflating liquid and supplies the inflating liquid between the balloon rear portion and the heart inner wall.

2. Heart inner wall checking tool of claim 1, wherein the shaft-shaped imaging member further comprises an imaging element on the distal end thereof, and is capable of imaging the heart inner wall from the inner rear portion of the inflated balloon.

3. Heart inner wall checking tool of claim 1, wherein the shaft-shaped imaging member further comprises optical fibers equipped with a lens on the distal end thereof and an imaging element on the proximal end thereof, and is capable of imaging the heart inner wall from the inner rear portion of the inflated balloon.

4. Heart inner wall checking tool of claim 1, wherein the balloon rear portion protrudes in the rear of a junction upper end provided between the balloon and the tubular main body when the balloon is inflated.

5. Heart inner wall checking tool of claim 1, wherein the liquid-exudable member is formed in a ring-shaped area having a certain width so as to surround the peripheral edge of an opening of the balloon.

6. Heart inner wall checking tool of claim 1, wherein the balloon comprises a rear-protruding-member forming member for forming the rear protruding member which protrudes in the rear of the junction upper end provided between the balloon and the tubular main body when the balloon is inflated.

7. Heart inner wall checking tool of any of claim 6, wherein the rear-protruding-member forming member is formed with a multiple number of linear members, one end of which is fixated onto the tubular main body; and the other end of which reaches the liquid-exudable member.

8. Heart inner wall checking tool of claim 6, wherein the rear-protruding-member forming member is formed with a ring-shaped member, which is fixated onto the tubular main body; as well as with a multiple number of linear members, one end of which is in the ring-shaped member; and the other end of which reaches the liquid-exudable member.

9. Heart inner wall checking tool of claim 1, wherein the liquid-exudable member has a numerous number of micropores, through which the inflating liquid is exuded.

10. Heart inner wall checking tool of claim 1, wherein the heart inner wall checking tool comprises a fixating member for fixating the heart inner wall checking tool onto the heart.

11. Heart inner wall checking tool of claim 1, wherein the heart inner wall checking tool comprises a hub provided on the proximal end of the tubular main body, and the hub is a branch hub comprising a liquid injection port and a shaft-shaped imaging member port.

12. Heart inner wall checking tool of claim 1, wherein the heart inner wall checking tool comprises a sheath which accommodates the balloon and the distal end of the tubular main body in a slidable manner.

13. Heart inner wall checking tool of claim 1, wherein the heart inner wall checking tool comprises a hub which is provided on the proximal end of the tubular main body, and the hub comprises a sealing member which renders the shaft-shaped imaging member to be slidable and rotatable under liquid tightness condition.

14. A heart inner wall checking device comprising the heart inner wall checking tool of claim 1 and a shaft-shaped imaging member which can be inserted into the heart inner wall checking tool or minimally the distal end of which is accommodated within the heart inner wall checking tool; wherein the shaft-shaped imaging member is capable of photographing the heart inner wall from the inner rear side of the inflated balloon.

15. Heart inner wall checking device of claim 14, wherein the shaft-shaped imaging member comprises a shaft-shaped main body; an imaging element, which is provided on the distal end of the shaft-shaped main body; and a connector, which is electrically connected to the imaging element.

16. Heart inner wall checking device of claim 15, wherein the shaft-shaped imaging member comprises the imaging element, which is provided so as to face forward, and a mirror, which enables the imaging element to image the targets in the diagonal rear direction.

17. Heart inner wall checking device of claim 15, wherein the shaft-shaped imaging member comprises the imaging element, which is provided so as to face the diagonal rear direction.

18. Heart inner wall checking device of claim 14, wherein the shaft-shaped imaging member comprises optical fibers equipped with a lens on the distal end thereof, and an imaging element provided on the proximal end of the optical fibers.

19. Heart inner wall checking device of claim 14, wherein the heart inner wall checking tool comprises a hub provided on the proximal end of the tubular main body, and the hub comprises a sealing member which renders the shaft-shaped imaging member to be slidable and rotatable under liquid tightness condition.

20. Heart inner wall checking device of claim 14, wherein the shaft-shaped imaging member can be inserted through the imaging member port.

21. Heart inner wall checking device of claim 14, wherein the shaft-shaped imaging member comprises an operating grip member provided on the proximal end thereof.

22. Heart inner wall checking device of claim 21, wherein the shaft-shaped imaging member, excluding the proximal end thereof, is accommodated within the heart inner wall checking tool, and the operating grip member of the shaft-shaped imaging member is equipped with an engaging member, which is provided on the rear end of the hub of the heart inner wall checking tool in a non-removable manner and in a manner movable in a certain longitudinal direction.
